(19) United States
(12) Patent Application Publication
(10) Pub. No.: US 2007/0123455 A1
(43) Pub. Date: May 31, 2007

(54) Immunomodulatory agents for treatment of inflammatory diseases

(76) Inventors: Joel Palefsky, Redwood City, CA (US); Herve Sroussi, Blencoe (IL)

Correspondence Address: Medlen & Carroll, 101 Howard Street, Suite 350, San Francisco, CA, 94105, US

(21) Appl. No.: 10/551,704

(22) PCT Filed: Apr. 2, 2004
(86) PCT No.: PCT/US04/10244

§ 371 Date: Jan. 22, 2007

Related U.S. Application Data
(60) Provisional application No. 60/460,652, filed on Apr. 4, 2003.

(51) Int. Cl. A61K 38/17; A61K 38/55

(52) U.S. Cl. 514/12

(57) ABSTRACT

The present invention provides methods and compositions suitable for treating inflammatory disorders such as allergy, asthma, artherosclerosis, autoimmune disease, infection, injury, meningitis, psoriasis, and transplant rejection. In particular, the present invention provides methods and compositions comprising human S100A8 and/or S100A9 for reducing inflammation.

This application claims the benefit of U.S. Provisional Application No. 60/460,652, filed on Apr. 4, 2004.

The invention was made in part with Government support by the National Institutes of Health, National Institute of Dental and Craniofacial Research, Grants 5P01DE007946, and 5P50DE011912. As such, the Government has certain rights in the invention.

### FIELD OF THE INVENTION

The present invention provides methods and compositions suitable for treating inflammatory disorders such as allergy, asthma, artherosclerosis, autoimmune disease, infection, injury, meningitis, psoriasis, and transplant rejection. In particular, the present invention provides methods and compositions comprising human S100A8 and/or S100A9 for reducing inflammation.

### BACKGROUND OF THE INVENTION

Inflammation is a localised protective response elicited by injury or destruction of tissues, which serves to destroy, dilute or sequester both the injurious agent and the injured tissue. It is characterized by pain (dolor), heat (calor), redness (rubor), swelling (tumour) and loss of function (functio laesa). Histologically, it involves a complex series of events, including dilatation of arterioles, capillaries and venules, with increased permeability and blood flow, exudation of fluids, including plasma proteins and leukocytic migration into the inflammatory focus. Although, generally protective in nature, uncontrolled or dysregulated inflammatory responses can be profoundly harmful.

For instance, asthma is a chronic lung condition with symptoms of difficulty breathing and wheezing caused by inflammation and narrowing of the air passages. The flow of air is blocked partially or completely as mucus produced by the inflammation fills a narrower passageway. Asthma affects both the lung's larger airways, called the bronchi, and the lung's smaller airways, called the bronchioles. Treatment focuses on preventing or stopping the inflammation, and relaxing the muscles that line the airways. What causes asthma-related inflammation is not clear, but several environmental “triggers” (e.g. allergens, viral infections, environmental pollutants, etc.) have been identified. Asthma has a genetic (inherited) component and often affects people with a family history of allergies. To date, more than 100 million people worldwide are estimated to have asthma, including between 12 and 15 million US citizens. Importantly, the incidence rate has increased by almost 50 percent since the early 1980s, indicating that this important medical problem has not been solved.

Although, there is no real cure, there are several different types of medications for the treatment of asthma, categorized as bronchodilators, anti-inflammatories and immunotherapies. Bronchodilators (e.g., beta agonists and xanthines) relax the muscles around the airways to improve airflow, and are commonly inhaled. In contrast, anti-inflammatories (e.g., steroidal and non-steroidal anti-inflammatories, and leukotrienes) work by reducing inflammation in the lung. Some people with mild to moderate symptoms of asthma also benefit from immunotherapy, in which the person is injected with increasing amounts of allergens to desensitize his or her immune system. Severe asthma attacks, on the other hand, must be treated in a hospital, where oxygen can be administered, and drugs may be given either intravenously or with a nebulizer.

Unfortunately, none of the current treatments for inflammatory disorders is ideal. For instance, the long-term use of oral or injected corticosteroids may result in poor wound healing, stunted growth in children, loss of calcium from the bones, stomach bleeding, and/or other problems. Similarly, non-steroidal anti-inflammatories may cause life-threatening ulcers after long term use. Thus, there is a need in the art for new medicines to treat inflammation.

### SUMMARY OF THE INVENTION

The present invention provides methods and compositions suitable for treating inflammatory disorders such as allergy, asthma, artherosclerosis, autoimmune disease, infection, injury, meningitis, psoriasis, and transplant rejection. In particular, the present invention provides methods and compositions comprising human S100A8 and/or S100A9 for reducing inflammation.

In particular, the present invention provides compositions comprising a nucleic acid sequence encoding a mutant human S100A8 or S100A9 protein, wherein the nucleic acid sequence comprises at least one mutation inhibiting post-translational modification of said protein. In some embodiments, inhibiting the post-translational modification comprises conferring oxidation resistance to the protein. In preferred embodiments, the mutation further prevents dimerization of the protein. In some embodiments, the mutation results in an amino acid substitution of a cysteine, a lysine or a methionine residue, and the mutation does not destroy the leukocyte-repellent activity of the protein. In a particularly preferred embodiment, the amino acid substitution comprises a replacement of Cysteine at residue 42 with an Alanine in the human S100A8 protein. In another preferred embodiment, the amino acid substitution comprises a replacement of Methionine at one or more of residue 61, residue 81, and residue 83, with an Alanine in the human S100A9 protein.

Also provided by the present invention are compositions, comprising a mutant human S100A8 or S100A9 protein, wherein the protein comprises at least one mutation inhibiting posttranslational modification of the protein. In some embodiments, inhibiting posttranslational modification comprises conferring oxidation resistance to the protein. In preferred embodiments, the mutation further prevents dimerization of the protein. In some embodiments, the mutation results in an amino acid substitution of a cysteine, a lysine or a methionine residue, and the mutation does not destroy the leukocyte-repellent activity of the protein. In a particularly preferred embodiment, the amino acid substitution comprises a replacement of Cysteine at residue 42 with an Alanine in the human S100A8 protein. In another preferred embodiment, the amino acid substitution comprises a replacement of Methionine at one or more of residue 61, residue 81, and residue 83, with an Alanine in the human S100A9 protein.

In addition, the present invention provides methods comprising: providing; i) at least one leukocyte, and ii) a composition comprising a human S100A8 or S100A9 protein; and contacting the leukocyte with the composition under conditions suitable for repelling the leukocyte. In some embodiments, the leukocyte is selected from but not limited to a peripheral monocyte, a neutrophil and an eosinophil. In some preferred embodiments, the leukocyte expresses at least one chemokine receptor selected from the group including but not limited to CCR1, CCR3 and CCR5. Moreover, in some embodiments, the protein comprises at least one mutation inhibiting posttranslational modification of said protein. In some preferred embodiments, inhibiting posttranslational modification comprises conferring oxidation resistance to the protein. In related embodiments, the mutation further prevents dimerization of the protein.

The present invention also provides methods comprising: providing; i) a subject with one or more symptoms of inflammation; and ii) a composition comprising a human S100A8 or S100A9 protein; and administering the composition to the subject under conditions such that at least one of the symptoms is reduced or eliminated. In preferred embodiments, the subject has an inflammatory disorder selected from but not limited to allergy, asthma, artherosclerosis, atopic dermatitis, autoimmune disease, cystic fibrosis, infection, injury, meningitis, psoriasis, and transplant rejection. In some embodiments, the infection is with a microorganism selected from but not limited to Candida albicans, Pseudomonas aeriginosa, human papillomavirus-16, and human immunodeficiency virus type 1. In related embodiments, the one or more symptoms are selected from the group including but not limited to pain, heat, redness and swelling. In some embodiments, swelling comprises a leukocyte infiltrate, which in preferred embodiments, comprises a cell selected from but not limited to a monocyte, a neutrophil, and an eosinophil. In particularly preferred embodiments, the protein comprises at least one mutation inhibiting posttranslational modification of the protein. In preferred embodiments, inhibiting posttranslational modification comprises conferring oxidation resistance. In related embodiments, the mutation further prevents dimerization of the protein.

Furthermore, the present invention provides methods comprising: providing: i) at least one cell expressing at least one chemokine receptor, and ii) a composition comprising a human S100A8 or S100A9 protein comprising a modification; and contacting the cell with the composition under conditions suitable for inhibiting the chemokine receptor. In some embodiments, the cell is selected from the group but not limited to a peripheral monocyte, a neutrophil, and an eosinophil. In preferred embodiments, the chemokine receptor is selected from the group including but not limited to CCR1, CCR3, and CCR5. In some particularly preferred embodiments, the modification is selected from the group consisting of oxidation and mutation. The present invention provides embodiments in which inhibiting the chemokine receptor comprises the binding of the protein to the receptor without activating the receptor. In some embodiments, the inhibiting is detected by a reduction in a chemokine-induced increase in cell size. In related embodiments, the reduction is measured by flow cytometry, and the chemokine is selected from the group including but not limited to MIP-1α, RANTES, eotaxin-1, eoxtaxin-2, eotaxin-3, MCP-2, MCP-3, MCP-4, and MIP-5.

### DESCRIPTION OF THE FIGURES

FIG. 1 provides the results from under-agarose migration studies using human peripheral blood monocytes (PM). Each bar represents the average±standard error of the mean of three experiments conducted in duplicate. Panel A depicts the influence of human S100A8 and S100A9 on migration. The columns represent: 1) control; 2) MCP1 10⁻⁹ mol; 3) MCP1 10⁻¹¹ mol; 4) MCP1 10⁻¹² mol; 5) MCP1 10⁻¹³ mol; 6) S100A8 10⁻¹² mol; 7) S100A8 10⁻¹³ mol; 8) S100A8 10⁻¹⁴ mol; 9) S100A9 10⁻¹² mol; 10) S100A9 10⁻¹³ mol; and 11) S100A9 10⁻¹⁴ mol. Panel B depicts the effect of potential modulators on human S100A8 and S100A9-induced PM fugetaxis. The effect of 10^{-12mol} of S100A8 and S100A9 was tested with and without the addition of 10^{-12mol} of different chemokines to the PM-containing well. The columns represent: 1) S100A8/RANTES; 2) S100A9/RANTES; 3) S100A8/MCP1; 4) S100A9/MCP1; 5) S100A8/IL-8; 6) S100A9/IL-8; 7) S100A8/anti-CCR3 Ab; 8) S100A9/anti-CCR3 Ab; 9) S100A8/anti-CD4 Ab; and 10) S100A9/anti-CD4 Ab.

FIG. 2 provides the results from transwell chemotaxis assays using wild type and chemokine-receptor transfected 4DE4 cells. Panel A depicts the influence of chemokine receptor expression on human S100A8 and S100A9 induced migration. The columns represent: 1) control; 2) eotaxin 10⁻⁸ mol; 3) MIP1α 10⁻⁸ mol; 4) IL-8 10⁻⁸ mol; 5) MCP1 10⁻⁸ mol; 6) S100A8 10⁻⁹ mol; 7) S100A8 10⁻¹⁰ mol; 8) S100A9 10⁻⁹ mol; and 9) S100A9 10⁻¹⁰ mol. Panel B depicts the results of checkerboard migration assays.

FIG. 3 provides polyacrylamide gel electrophoretic analyses of mutant human S100A8 protein, and an analysis of its effect on chemokine-receptor mediated chemotaxis and neutrophil fugetaxis. Panel A depicts a Coomassie blue-stained nonreduced gel. The contents of the lanes are as follows: 1) wild type S100A8; and 2) thrombin-cleaved mutant Ala⁴²S100A8 fusion protein. Panel B depicts a western blot analysis of recombinant S100A8 proteins detected with a human S100A8-reactive mouse mAb (8-5C2). The mutant human Ala⁴²S100A8 fusion protein was run in lane 1, while the wild type human S100A8 was run in lane 2. Panel C shows that like the wild type human protein, that the mutant human Ala⁴²S100A8 protein is a chemoattractant for CCR1- and CCR3-transfected cells. Panel D shows that the fugetaxic effect of the mutant human Ala⁴²S100A8 protein, is resistant to oxidation, whereas the wild type human S100A8 protein's fugetaxic effect is inhibited by oxidation.

FIG. 4 provides the results from a transwell migration assays and a shape change assay using human peripheral granulocytes. The data represent the average of four experiments performed in duplicate. Panel A depicts the influence of human S100A8 (10⁻⁹M) on the migration of peripheral granulocytes. The columns represent: 1) control; 2) S100A8 in the upper chamber; 3) S100A8 in the lower chamber; and 4) S100A8 in both the upper and lower chambers. Panel B depicts the influence of IL-8 and mutant human Ala⁴²S100A8 at 10⁻⁹M on the migration of peripheral granulocytes. The columns represent: 1) control (no chemokine); 2) IL-8 in the lower chamber; 3) Ala⁴²S100A8 in the upper chamber; 4) Ala⁴²S100A8 in the lower and upper chambers; and 5) Ala⁴²S100A8 in the lower chamber. Panel C depicts the influence of various cytokines on the shape of peripheral granulocytes. Cell size was measured with the forward scatter channel of a flow cytometer and indicated as a percentage of the basal level.

FIG. 5 depicts the effect of Ala⁴²S100A8 on lipopolysaccharide (LPS)-induced recruitment of leukocytes in a rat air-pouch model as measured by the number of neutrophils recruited into the air pouch. Panel A indicates that co-injection of Ala⁴²S100A8 inhibited the inflammatory response induced by LPS. Panel B provides a dose-response curve of Ala⁴²S100A8 inhibition of neutrophil recruitment.

FIG. 6 panel A depicts the cDNA sequence of human S100A8 (SEQ ID NO:1), while panel B depicts the amino acid sequence of S100A8 (SEQ ID NO:2).

FIG. 7 panel A depicts the cDNA sequence of human S100A9 (SEQ ID NO:3), while panel B depicts the amino acid sequence of S100A9 (SEQ ID NO:4).

FIG. 8 depicts the influence of human S100A8 and S100A9 on eotaxin-induced migration of CCR3-transfected cells in a transwell migration assay. Eotaxin (10⁻⁹M) was added to the lower well and various chemokines were added to the upper well at equal concentrations. The columns represent: 1) control; 2) eotaxin; 3) oxidized wild-type S100A8; 4) S100A9; and 5) MCP1.

FIG. 9 illustrates that S100A8/A9 complexes inhibit Candida albicans growth. Panel A depicts the effect of wild type and mutant complexes containing S100A9 variants on the growth of Candida. Panel B illustrates that cysteine at residue 42 of S100A8 is required for Candida inhibition.

FIG. 10 illustrates that S100A9 promotes Pseudomonas aeruginosa growth. Panel A illustrates the enhancement of Pseudomonas growth in the presence of S100A9. The data are shown in duplicate, and indicate that S100A9 treatment results in a shortening of the lag phase and an acceleration of the growth phase of Pseudomonas. Panel B illustrates that mutant S100A9 proteins inhibit wild type S100A-enhanced growth of Pseudomonas. The 61ALA S100A9 mutant displays a dominant-negative effect over the wild type S100A9 protein.

### DEFINITIONS

To facilitate an understanding of the present invention, a number of terms and phrases are defined below:

The term “gene” refers to a nucleic acid (e.g., DNA) sequence that comprises coding sequences necessary for the production of a polypeptide or precursor or RNA (e.g., tRNA, siRNA, rRNA, etc.). The polypeptide can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the sequences located adjacent to the coding region on both the 5′ and 3′ ends, such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region, which may be interrupted with non-coding sequences termed “introns” or “intervening regions” or “intervening sequences.” Introns are removed or “spliced out” from the nuclear or primary transcript, and are therefore absent in the messenger RNA (mRNA) transcript. The mRNA functions during translation to specify the sequence or order of amino acids in a nascent polypeptide.

In particular, the term “S100A8 gene” refers to the full-length S100A8 nucleotide sequence (e.g., SEQ ID NO:1). However, it is also intended that the term encompass fragments of the S100A8 nucleotide sequence, as well as other domains (e.g., functional domains) within the full-length S100A8 nucleotide sequence. For example, the term encompasses nucleic acid sequences that are from 50%, 60%, 70%, 80%, 90%, to 95% or more identical to SEQ ID NO:1. The term also encompasses nucleic acid sequences that encode an S100A8 protein (e.g., SEQ ID NO:2) and sequences that are from 50%, 60%, 70%, 80%, 90%, to 95% or more identical to SEQ ID NO:2. In each case, the S100A8 sequences preferably encode a protein that upregulates fugetaxis. Furthermore, the terms “S100A8 gene,” “S100A8 nucleotide sequence,” and “S100A8 polynucleotide sequence” encompass DNA, cDNA, and RNA sequences.

The term “plasmid” as used herein, refers to a small, independently replicating, piece of DNA. Similarly, the term “naked plasmid” refers to plasmid DNA devoid of extraneous material typically used to affect transfection. As used herein, a “naked plasmid” refers to a plasmid substantially free of calcium-phosphate, DEAE-dextran, liposomes, and/or polyamines.

As used herein, the term “purified” refers to molecules (polynucleotides or polypeptides) that are removed from their natural environment, isolated or separated. “Substantially purified” molecules are at least 50% free, preferably at least 75% free, and more preferably at least 90% free from other components with which they are naturally associated.

The term “recombinant DNA” refers to a DNA molecule that is comprised of segments of DNA joined together by means of molecular biology techniques. Similarly, the term “recombinant protein” refers to a protein molecule that is expressed from recombinant DNA.

The term “fusion protein” as used herein refers to a protein formed by expression of a hybrid gene made by combining two gene sequences. Typically this is accomplished by cloning a cDNA into an expression vector in frame with an existing gene. The fusion partner may act as a reporter (e.g., βgal) or may provide a tool for isolation purposes (e.g., GST).

Suitable systems for production of recombinant proteins include but are not limited to prokaryotic (e.g., Escherichia coli), yeast (e.g., Saccaromyces cerevisiae), insect (e.g., baculovirus), mammalian (e.g., Chinese hamster ovary), plant (e.g., safflower), and cell-free systems (e.g., rabbit reticulocyte).

As used herein, the term “coding region” refers to the nucleotide sequences that encode the amino acid sequences found in the nascent polypeptide as a result of translation of an mRNA molecule. The coding region is bounded in eukaryotes, on the 5′ side by the nucleotide triplet “ATG” that encodes the initiator methionine and on the 3′ side by one of the three triplets which specify stop codons (i.e., TAA, TAG, and TGA).

Where amino acid sequence is recited herein to refer to an amino acid sequence of a naturally occurring protein molecule, “amino acid sequence” and like terms, such as “polypeptide” or “protein,” are not meant to limit the amino acid sequence to the complete, native amino acid sequence associated with the recited protein molecule.

The term “wild-type” refers to a gene or gene product that has the characteristics of that gene or gene product when isolated from a naturally occurring source. A wild type gene is that which is most frequently observed in a population and is thus arbitrarily designed the “normal” or “wild-type” form of the gene.

As used herein, the terms “mutant,” “polymorphism,” and “variant,” in reference to a gene or gene product, refer to alterations in sequence and/or functional properties (i.e., different characteristics) when compared to the wild-type gene or parental gene product. In some preferred embodiments, the term mutant refers to a gene or gene product that differs from a parental gene or gene product as a result of mutation. It is noted that naturally occurring and induced mutants can be isolated; these are identified by the fact that they have altered characteristics when compared to the wild-type gene or parental gene product. In addition, mutant genes can be artificially (e.g., site-directed mutagenesis) or synthetically produced in the laboratory.

The term “mutation” refers to a change in the number, arrangement, or molecular sequence of nucleotides in a genetic sequence. In other embodiments “mutation” refers to a change in number, arrangement, or a specified amino acid sequence of a peptide or a protein.

As used herein in reference to a protein, the term “modified” refers to proteins with structural changes including primary, secondary, tertiary, etc. changes. Thus, the term “modified” encompasses but is not limited to amino acid deletions, insertions and substitutions (e.g., as a result of mutation), as well as post-translational modifications such as glycosylation, acylation, limited proteolysis, phosphorylation, isoprenylation and oxidation. In addition, the term modified encompasses the replacement of a native amino acid with a non-standard residue including but not limited to acetamidomethyl, aminohexanoic acid, aminoisobutyric acid, beta-alanine, cyclohexylalanine, D-cyclohexylalanine, e-acetyl lysine, gamma aminobutyric acid, hydroxyproline, nitro-arginine, nitro-phenylalanine, nitro-tyrosine, norleucine, norvaline, octahydroindole carboxylate, ornithine, penicillamine, phenylglycine, phosphoserine, phosphothreonine, phosphotyrosine, pyroglutamate, and tetrahydroisoquinoline.

The terms “S100A8,” “cystic fibrosis antigen,” “calprotectin,” “calgranulin A,” “L1 light chain,” “CP-10” and “MCP8,” as used herein refer to a human S100A8 gene (e.g., Homo sapiens—GENBANK Accession No. NM 002964) and its gene product (GENBANK Accession No. NP_{—}002955.2), including wild type and mutant products. Human S100A8 variants, which differ from the wild type S100A8 sequences in fewer than 1% of the residues, are also suitable for use in the methods and compositions of the present invention.

As used herein, the terms “S100A9,” “calgranulin B,” “L1 heavy chain,” and “MCP 14” refer to a human S100A9 gene (e.g., Homo sapiens—GENBANK Accession No. NM_{—}002965) and its gene product (GENBANK Accession No. NP_{—}002956.1), including wild type and mutant products. Human S100A9 variants, which differ from the wild type S100A9 sequences in fewer than 1% of the residues, are also suitable for use in the methods and compositions of the present invention.

The term “antisense molecule” refers to polynucleotides and oligonucleotides capable of binding to an mRNA molecule. In particular, an antisense molecule is a DNA or RNA sequence complementary to an mRNA sequence of interest. In preferred embodiments, the term S100 antisense molecule refers to a single-stranded DNA or RNA sequence that binds to at least a portion of an S100 mRNA molecule to form a duplex which then blocks further transcription and/or translation.

As used herein, the terms “complementary” and “complementarity” refer to polynucleotides related by base-pairing rules. For example, for the sequence “5′-AGT-3′,” the complementary sequence is “3′-TCA-5′.”

The term “antibody” refers to polyclonal and monoclonal antibodies. Polyclonal antibodies which are formed in the animal as the result of an immunological reaction against a protein of interest or a fragment thereof, can then be readily isolated from the blood using well-known methods and purified by column chromatography, for example. Monoclonal antibodies can also be prepared using known methods (See, e.g., Winter and Milstein, Nature, 349, 293-299, 1991). As used herein, the term “antibody” encompasses recombinantly prepared, and modified antibodies and antigen-binding fragments thereof, such as chimeric antibodies, humanized antibodies, multifunctional antibodies, bispecific or oligo-specific antibodies, single-stranded antibodies and F(ab) or F(ab)₂ fragments. The term “reactive” in used in reference to an antibody indicates that the antibody is capable of binding an antigen of interest. For example, an S100A8-reactive antibody is an antibody, which binds to S100A8 or to a fragment of S100A8.

The term “portion” when used in reference to a nucleotide sequence refers to fragments of that sequence, which range in size from 10 nucleotides to the entire nucleotide sequence minus one nucleotide.

As used herein, the term “biologically active” refers to a molecule having structural, regulatory and or biochemical functions of a wild type homeobox molecule. In some instances, the biologically active molecule is a homolog of a mammalian homeobox molecule, while in other instance the biologically active molecule is a portion of a mammalian homeobox molecule. Other biologically active molecules, which find use in the compositions and methods of the present invention include but are not limited to mutant (e.g. variants with at least one deletion, insertion or substitution) mammalian S100 molecules. Biological activity is determined for example, by restoration or introduction of S100 (e.g., S100A9 or S100A8) activity in cells which lack S100 activity, through transfection of the cells with a S100 expression vector containing a S100 gene, derivative thereof, or portion thereof. Methods useful for assessing S100A8 and S100A9 activity include but are not limited to transwell migration assays.

The term “conservative substitution” as used herein refers to a change that takes place within a family of amino acids that are related in their side chains. Genetically encoded amino acids can be divided into four families: (1) acidic (aspartate, glutamate); (2) basic (lysine, arginine, histidine); (3) nonpolar (alanine, valine, leucine, isoleucine, proline, phenylalanine, methionine, tryptophan); and (4) uncharged polar (glycine, asparagine, glutamine, cysteine, serine, threonine, tyrosine). Phenylalanine, tryptophan, and tyrosine are sometimes classified jointly as aromatic amino acids. In similar fashion, the amino acid repertoire can be grouped as (1) acidic (aspartate, glutamate); (2) basic (lysine, arginine, histidine), (3) aliphatic (glycine, alanine, valine, leucine, isoleucine, serine, threonine), with serine and threonine optionally be grouped separately as aliphatic-hydroxyl; (4) aromatic (phenylalanine, tyrosine, tryptophan); (5) amide (asparagine, glutamine); and (6) sulfur -containing (cysteine and methionine) (e.g., Stryer [ed.], Biochemistry, pg. 17-21, 2nd ed, WH Freeman and Co., 1981). Whether a change in the amino acid sequence of a peptide results in a functional homolog can be readily determined by assessing the ability of the variant peptide to function in a fashion similar to the wild-type protein. Peptides having more than one replacement can readily be tested in the same manner. In contrast, the term “nonconservative substitution” refers to a change in which an amino acid from one family is replaced with an amino acid from another family (e.g., replacement of a glycine with a tryptophan). Guidance in determining which amino acid residues can be substituted, inserted, or deleted without abolishing biological activity can be found using computer programs (e.g., LASERGENE software, DNASTAR Inc., Madison, Wis.).

As used herein the term “animal” refers to any member of the kingdom Animalia, which includes living things, which have cells differing from plant cells with regard to the absence of a cell wall and chlorophyll and the capacity for spontaneous movement. Preferred embodiments of the present invention are primarily directed to vertebrate (backbone or notochord) members of the animal kingdom.

The terms “patient” and “subject” refer to a mammal or an animal who is a candidate for receiving medical treatment.

The term “control” refers to subjects or samples which provide a basis for comparison for experimental subjects or samples. For instance, the use of control subjects or samples permits determinations to be made regarding the efficacy of experimental procedures. In some embodiments, the term “control subject” refers to animals, which receive a mock treatment (e.g., βgal plasmid DNA).

As used herein, the terms “gene transfer” and “transfer of genetic information” refer to the process of moving a gene or genes from one place to another. In preferred embodiments of the present invention, the term “gene transfer” refers to the transfer of a polynucleotide to cells and/or tissues of an animal to achieve a therapeutic effect. In some embodiments, the polynucleotide may be in the form of a plasmid, a gene fragment or an oligonucleotide. In some embodiments, “gene transfer” is temporary or transient, in other embodiments “gene transfer” is sustained, and in still further embodiments, the gene transfer is long-lived, permanent or stable.

As used herein, “gene transfer” may affect the transfection of cells and/or tissues. The term “transfection” refers to the introduction of foreign DNA into eukaryotic cells.

As used herein, the terms “localized” and “local” refer to the involvement of a limited area. Thus, in contrast to “systemic” treatment, in which the entire body is involved, usually through the vascular and/or lymph systems, localized treatment involves the treatment of a specific, limited area. Thus, in some embodiments, discrete wounds are treated locally using the methods and compositions of the present invention.

As used herein, the term “topically” means application to the surface of the skin, mucosa, viscera, etc. Similarly, the terms “topically active drug” and “topically active agent” refer to a substance or composition, which elicits a pharmacologic response at the site of application (e.g., skin), but is not necessarily an antimicrobial agent.

As used herein, the terms “systemically active drug” and “systemically active agent” are used broadly to indicate a substance or composition that will produce a pharmacologic response at a site remote from the point of application.

The terms “sample” and “specimen” are used in their broadest sense. On the one hand, they are meant to include a specimen or culture. On the other hand, they are meant to include both biological and environmental samples. These terms encompasses all types of samples obtained from humans and other animals, including but not limited to, body fluids such as urine, blood, fecal matter, cerebrospinal fluid, semen, saliva, and wound exudates, as well as solid tissue. However, these examples are not to be construed as limiting the sample types applicable to the present invention.

As used herein, the term “oxidation resistance” refers to the ability to resist the loss of electrons from oxidation.

The term “dimerization” and alternatively “dimerize, as used herein refers to the ability to form a structure from the association of two subunits.

As used herein, the term “amino acid substitution” refers to an act, process, or result of substituting amino acids.

The term “leukocyte” as used herein, refers to cells called white blood cells, that help the body fight infections and other diseases, and include for instance granulocytes (e.g., neutrophils, eosinophils, basophils), monocytes, and lymphocytes (e.g., B cells, T cells, natural killer cells).

As used herein, the term “monocyte” refers to a mononuclear phagocyte circulating in blood that will later emigrate into tissue and differentiate into a macrophage.

The terms “neutrophil,” and “major circulating phagocytic polymorphonuclear granulocyte,” refer to a type of white blood cell characterized by secondary granules that stain pink with Wright or Giemsa stains and which constitute approximately 60% of the blood in a healthy individual.

As used herein, the term “chemokine” refers to soluble factors (e.g., cytokines) that have the ability to selectively induce chemotaxis and activation of leukocytes. They also trigger integrin-mediated leukocyte activation, and attract leukocytes to places where they are needed (e.g., sites of inflammation or infection). Currently, the chemokine superfamily is separated into three distinct subfamilies called Alpha (or C-X-C), Beta (C-C), and Gamma (C). These designations are based upon the positioning of their cysteine residues. The Alpha chemokines have a single amino acid inserted between the first and second of their four cysteine residues, hence C-X-C, whereas these cysteines are not separated in the Beta group, hence C-C, while the gamma (C) chemokines have only one pair of cysteines.

As used herein, the terms “CCR1” and “CC-chemokine receptor 1,” refer to a member of the human CC chemokine receptor family. CCR1 has the nonexclusive ability to bind to RANTES (regulated upon activation normal T-expressed and presumably secreted), macrophage inflammatory protein 1 alpha (MIP-1 alpha), monocyte chemoattractant protein 2 (MCP-2) and monocyte chemoattractant protein 3 (MCP-3).

The terms “CCR3” and “CC-chemokine receptor 3,” as used herein, refer to a member of the human CC chemokine receptor family. CCR3 has the nonexclusive ability to bind to eotaxin, eotaxin-2, eotaxin-3, RANTES (regulated upon activation normal T-expressed and presumably secreted), monocyte chemoattractant protein 2 (MCP-2), monocyte chemoattractant protein 3 (MCP-3) and monocyte chemoattractant protein 4 (MCP-4).

As used herein, the terms “CCR5” and “CC-chemokine receptor 5,” refer to a member of the human CC chemokine receptor family. CCR5 has the nonexclusive ability to bind to RANTES (regulated upon activation normal T-expressed and presumably secreted), macrophage inflammatory protein 1 alpha (MIP-1α) and macrophage inflammatory protein 1 beta (MIP-1β).

The terms “chemokine receptor” and “serpentine receptor” as used herein, refer to a G protein-linked receptors which are chemokine ligands having seven membrane spanning domains. Chemokine receptors are found on the surface of leukocytes and other cell types, and exhibit some degeneracy in chemokine ligand binding. For example, the low affinity IL-8R also binds the related GRO and NAP-2 chemokines, and the MIP-1 alpha receptor also binds RANTES.

As used herein, the terms “IL-8,” “neutrophil-activating peptide 1,” and “NAP-1,” refer to interleukin-8, a CXC chemokine that acts as a chemoattractant for neutrophils and basophils.

The term “eotaxin” as used herein, refers to a CC chemokine, which is a powerful attractant for eosinophils.

As used herein, the terms “RANTES” and “regulated-upon-activation, normal T expressed and secreted” refer to a CC chemokine that is produced by T cells and platelets, and acts as a chemoattractant for monocytes, some T lymphocytes and eosinophils.

The term “chemotaxis” as used herein refers to a response of motile cells in which the direction of movement is toward the direction of a gradient (e.g., up) of a diffusible substance. This differs from “chemokinesis” in that the gradient alters probability of motion in one direction only, rather than rate or frequency of random motion.

As used herein, the terms “fugetaxis” and “anti-chemotaxis” refer to a response of motile cells in which the direction of movement is away from the gradient (e.g., down) of a diffusible substance.

The term “inflammation” as used herein, refers to the tissue response to trauma, characterized by increased blood flow and entry of leukocytes into the tissues, resulting in swelling, redness, elevated temperature and pain.

As used herein, the term “symptom” refers to any subjective evidence of disease or of a patient's condition (e.g., a change in a patient's condition indicative of some bodily or mental state).

For instance, the phrase “symptoms of inflammation” in the context of inflammatory bowel disease (IBD) is herein defined to include, but is not limited to symptoms such abdominal pain, diarrhea, rectal bleeding, weight loss, fever, loss of appetite, and other more serious complications, such as dehydration, anemia and malnutrition. A number of such symptoms are subject to quantitative analysis (e.g., weight loss, fever, anemia, etc.). Some symptoms are readily determined from a blood test (e.g., anemia) or a test that detects the presence of blood (e.g., rectal bleeding).

Similarly, the phrase “under conditions such that the symptoms are reduced” in the context of IBD refers to any degree of qualitative or quantitative reduction in detectable symptoms of IBD, including but not limited to, a detectable impact on the rate of recovery from disease (e.g., rate of weight gain), or the reduction of at least one of the following symptoms: abdominal pain, diarrhea, rectal bleeding, weight loss, fever, loss of appetite, dehydration, anemia, distention, fibrosis, inflamed intestines and malnutrition.

As used herein, the term “allergy” refers to a group of symptoms precipitated by an immune response to substances not typically triggering in an immune response in most individuals. Specific symptoms depend on the specific allergen (inciting substance), the body part exposed, and individual variation in immune responsiveness.

As used herein, the term “asthma” refers to a condition characterized by inflammatory constriction or congestion of the bronchial tree, causing wheezing, coughing and difficult breathing. Alternatively known as reactive airway disease.

As used herein, the term “arthritis” refers to an inflammatory condition that affects joints, which may be caused by, but not limited to, trauma (including long-term wear and tear), infection, or autoimmune responses.

As used herein, the term “atheroscelerosis” refers to a form of arteriosclerosis in which fatty deposits form on the inner walls of arteries and block the flow of blood.

As used herein, the term “autoimmune disease” refers to the pathological result when the immune system destructively attacks the body's own organs and tissues. An autoimmune disease may be the result of an amplification of naturally occurring reactivity that causes structural or functional pathology. The term autoimmune disease encompasses but is not limited to arthritis, diabetes, lupus, and multiple sclerosis.

As used herein, the term “infection” refers to invasion and reproduction of microorganisms in the body, which may be clinically undetectable or may result in tissue damage.

As used herein, the term “injury” refers to hurt, damage, or loss sustained of body tissue.

As used herein, the term “transplant rejection” refers to the immunological destruction of the transplanted tissue.

As used herein, the term “pain” refers to the usually localized physical suffering associated with bodily disorder (as in a disease or an injury). In particular, pain is a basic bodily sensation induced by a noxious stimulus, received by naked nerve endings, and characterized by physical discomfort (as pricking, throbbing, or aching).

As used herein, the term “heat” refers to becoming warm or hot (increased temperature). The term “redness” refers to the quality or state of being red. The term “swelling” refers to an expansion (as in size, volume, or numbers) beyond a normal or original limit. For example, tissue may become distended or puffed up, to form a bulge or rounded elevation due in part of leukocyte migration to the site of injury or tissue damage.

As used herein, the term “flow cytometry” refers to a laser-based machine and to the analysis of biological material by detection of the light-absorbing or fluorescing properties of cells or subcellular fractions (e.g., cells, cell lysates, etc.) passing in a narrow stream through a laser beam.

### DESCRIPTION OF THE INVENTION

The present invention provides methods and compositions suitable for treating inflammatory disorders such as allergy, asthma, artherosclerosis, autoimmune disease, infection, injury, meningitis, psoriasis, and transplant rejection. In particular, the present invention provides methods and compositions comprising human S100A8 and/or S100A9 for reducing inflammation.

As described in more detail below and in the experimental examples, multiple uses of human S100A8 and A9 have been identified. In preferred embodiments, the present invention provides compositions and methods comprising human S100A8 and A9 for upregulation of fugetaxis (e.g., as anti-inflammatories). In other embodiments, compositions and methods comprising oxidized human S100A8 and A9 for downregulation of fugetaxis are provided. In addition, compositions and methods comprising wild type, mutant or modified human S100A8 and A9 for blocking chemokine binding are provided.

I. Leukocyte Migration

The term leukocyte refers to white blood cells and includes granulocytes (e.g., basophils, eosinophils, and neutrophils), lymphocytes (e.g., B, T and NK cells), and monocytes. Leukocyte mobility is influenced by enhanced random migration (chemokinesis), or by introduction of a bias in the direction of cell movement up a chemical gradient (chemotaxis) (Wilkinson, FEMS Microbiol Immunol, 2:303-311, 1990). The movement of leukocytes from the circulation into extra-vascular spaces is orchestrated at least in part by chemoattractant molecules known as chemokines, which are categorized according to variations on a shared cysteine motif (Baggiolini et al., Annu Rev Immunol, 15:675-705, 1997). The effect of chemokines is concentration-dependent since at concentrations exponentially greater than their chemotactic concentrations, chemokines may also inhibit this migratory process or even repel leukocytes (Zlatopolskiy and Laurence, Immunol Cell Biol, 79:340-344, 2001). The process of retrograde cellular movements driven by chemokines has been termed “fugetaxis” (Poznansky et al., Nat Med, 6:543-548, 2000). Current evidence suggests that fugetaxis may be involved in thymocyte migration in vivo (Poznansky et al., J Clin Invest, 109:1101-1110, 2002). Recently it has been suggested that the migration of leukocytes up a gradient at low concentrations (chemotaxis) and down a gradient of the same molecules at higher concentrations (fugetaxis) is controlled not only by changes in chemokine concentration but also by intracellular signaling changes (Cyster, J Clin Invest, 109:1011-1012, 2002).

II. S100 Family of Calcium-Binding Proteins

S100 is a large evolutionarily-conserved subfamily of EF-hand calcium binding proteins. The basic structure of S100 proteins is as dimers, since S100 monomers are unstable. S100A8 and S100A9 also form calcium-dependent heterodimers and higher order species. The heterodimers are noncovalently bonded and are preferentially formed in solution over homodimers of each S100 protein. However upon oxidation, S100A8 preferentially homodimerizes following the formation of an intermolecular disulfide bridge between conserved cysteine residues (Harrison et al., J Biol Chem, 274:8561-8569, 1999).

S100A8 is also known as cystic fibrosis antigen (in complex with S100A9), p 23 (in complex with S100A9), calprotectin (in complex with S100A9), calgranulin A, L1 light chain, CP-10 and MCP-8. Similarly, S100A9 is also known as calgranulin B, L1 heavy chain, and MCP-14. More recently, the complex of S100A8 and S100A9 was shown to bind to arachidonic acid, the metabolites of which are involved in inflammation (Klempt et al., FEBS Lett, 408:81-84, 1997).

S100A8 and S100A9 lack a leader sequence or a transmembrane region and are secreted by a novel secretory pathway (Rammes et al., J Biol Chem, 272:9496-9502, 1997). These proteins are expressed in cells of the myeloid lineage and may represent up to 45% of the total cytosolic protein content of neutrophils (Edgeworth et al., J Biol Chem, 266:7706-7713, 1991). S100A8 and S100A9 are also produced in peripheral monocytes (PM) and tissues macrophages, as well as in epithelial cells under inflammatory conditions such as psoriasis (Nagpal et al., Cell Growth Differ, 7:1783-1791, 1996). Moreover, normal buccal mucosal epitheliual cells express S100A8 and S100A9 constitutively at low levels (Wilkinson et al., J Cell Sci, 91:221-230, 1988), whereas keratinized epithelium expresses the two proteins under conditions of stress (Marionnet et al., J Invest Dermatol, 121:1447-1458, 2003). S100A8 and S100A9 are also detectable in the circulation.

S100A8 and S100A9 are postulated to have chemoattractant properties based on several lines of evidence (Kerkhoff et al., Biochim Biophys Acta, 1448:200-211, 1998; and Passey et al., J Leukoc Biol, 66:549-556, 1999). The murine homologue of S100A8 is a strong chemoattractant for peripheral monocytes (PM) and neutrophils at an extremely low concentration (10⁻¹³ M) (Lackmann et al., J Immunol, 150:2981-2991, 1993). In addition, bovine S100A2 and S100A7 (psoriasin) are chemotactic for guinea pig eosinophils and for CD4+ T lymphocytes and neutrophils, respectively (Komada et al., Biochem Biophys Res Commun, 220:871-874, 1996; and Jinquan et al., J Invest Dermatol, 107:5-10, 1996).

Understanding the functions of human S100A8 and S100 A9 is important because they are detected at a high level in a wide variety of inflammatory conditions (Gabrielsen et al., J Am Acad Dermatol, 15:173-179, 1986) both locally in epithelium and in the circulation (Lugering et al., Clin Exp Immunol, 101:249-253, 1995; Lugering et al., Digestion, 56:406-414, 1995; and Muller et al., J Acquir Immune Defic Syndr, 7:931-939, 1994). However, studies of the chemoattractant properties of human S100A8 and S100A9 have yielded variable results. While some investigators have reported chemotactic activity for S100A8 (Ryckman et al., J Immunol, 170:3233-3242, 2003), others have been unable to demonstrate a similar effect. Some have attributed this discrepancy to the susceptibility of the proteins to oxidation (Roth et al., J Immunol, 171:5651, 2003), but this discrepancy could also be attributed differences in methodology used to produce and assay the proteins.

III. Influence of Human S100A8 and S100A9 on Leukocyte Migration

During development of the present invention, S100A8 and A9 were found to be heavily overexpressed in lesions from patients with hairy leukoplakia. Hairy leukoplakia is a benign hyperplastic lesion of the oral cavity, most often found in the setting of HIV-related immuno-suppression. It is characterized by high-level Epstein Barr virus replication and the absence of leukocyte infiltrates (Daniels et al., J Invest Dermatol, 89:178-182, 1987). In addition, the S100A8 null mouse is associated with lethal infiltration of the null fetus with heterozygous maternal leukocytes (Passey et al., J Immunol, 163:2209-2216, 1999).

The inventors have now characterized the chemotactic properties of human S100A8 and S100A9 in a controlled cell culture environment in two different in vitro assays (under-agarose assays and transwell assays). As described in detail in the experimental examples and in the accompanying figures, human S100A8 and S100A9 were found to have potent fugetactic properties. In particular, human S10A8 and S100A9 at concentrations from 0.01 to 1.0 picomol produced a dose-dependent fugetactic migratory bias in under agarose migration assays, whereas concentrations of S100A8 and S100A9 below 0.01 picomol did not bias the migration of monocytes in either direction. In contrast, the chemokine MCP1 was shown to elicit a chemotactic response at 0.1, 1.0 and 10 picomol, and a fugetactic response at 1 nanomol. In further studies, the induction of PM fugetaxis by S100A8 and S100A9 was found to be abrogated by RANTES and an anti-CCR3 mAb (7B11), but not by MCP1, IL8, or an isotype-matched anti-CD4 mAb.

IV. Influence of Human S100A8 and S100A9 on Chemokine Receptor Activation

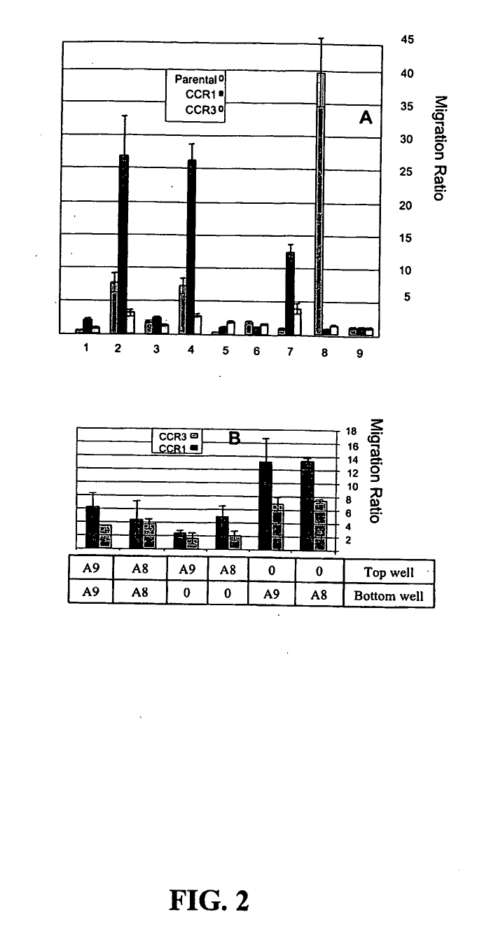

As described in detail in Example 3 below, and shown in FIG. 2, human S100A8 and S100A9 preferentially attracted CCR1+ cells and to a lesser degree CCR3+ cells in transwell chemotaxis assays. The control chemokines eotaxin and MIP1αattracted CCR3+ and CCR1+ cells respectively, in these same assays. A modest background attraction of the parental cells with S100A8 and S100A9, as well as with MIP1α, is contemplated to represent a previously unreported background effect on murine receptors. Moreover, oxidized wild type human S100A8 maintained its ability to inhibit eotaxin, despite losing its chemotactic activity on the CCR1+ and CCR3+transfected cells.

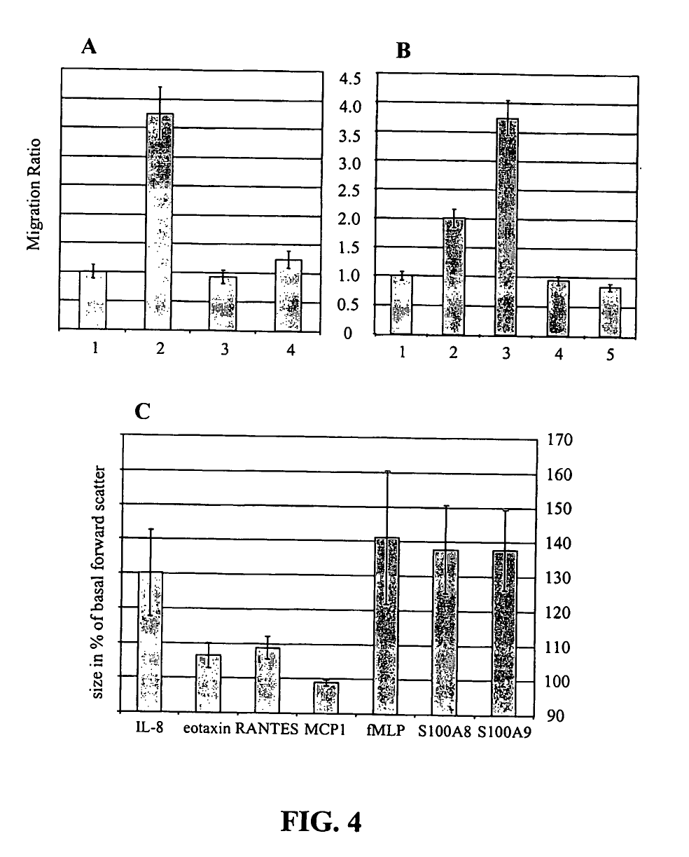

In addition, in transwell migration assays, S100A8 was found to exhibit a fugetactic effect on peripheral neutrophils (See, FIG. 4, panel A), and S100A9 was found to exert a similar effect on monocytes. Similarly, the mutant human Ala⁴²S100A8 protein also exhibited a fugetactic effect on periperhal neutrophils in transwell assays (See, FIG. 4, panel B), with a similar effect observed on monocytes. In shape change assays, like the positive control chemokines IL-8, eotaxin, RANTES, and fMLP, both human S100A8 and S100A9 caused a consistent increase in granulocyte cell size as indicated by an increase in forward scatter (See, FIG. 4, panel C). In contrast, the negative control MCP1 had no effect.

The human calcium-binding proteins S100A8 and S100A9 were shown herein for the first time to modulate leukocyte migration via the CCR1 and CCR3 chemokine receptors. Specifically, the activation of CCR1 and CCR3 by human S100A8 and S100A9 lead to a “fugetaxis only” response in PM and neutrophils. However, the present invention is not limited to methods and compositions comprising activation of CCR1 and CCR3. In fact, human S100A8 and S100A9 are contemplated to modulate leukocyte activity via any and all chemokine receptors to which RANTES and eotaxin binds, including for instance CCR5 and DARC (Duffy antigen receptor for chemokines), as well as additional chemokine receptors which have yet to be unidentified.

Moreover, at physiologically relevant concentrations, oxidation is contemplated to play a crucial role in the regulation of S100 function. Indeed, complete inactivation of the human S100 proteins by oxidation was observed, which is contemplated to occur through disulfide bond-mediated dimerization. Thus, expression of S100A8 and S100A9 in epithelium is contemplated to create a chemical barrier to inflammatory infiltrates. Oxidative modification of the S100 proteins by neutrophil enzymes secreted during acute inflammation is further contemplated to attenuate this barrier, allowing leukocyte translocation into extravascular spaces.

V. Human S100A8 and S100A9 as Immuno-Modulators

Human S100A8 and S100A9 are contemplated to play important roles in the pathogenesis and therapy of several immune-mediated diseases. In particular, RANTES has been shown to inhibit HIV infection via its interaction with the HIV co-receptor, CCR5, on a patient's target cells (Simmons et al., Immunol Rev, 177:112-126, 2000; and Proudfoot et al., Biochem Pharmacol, 57:451-463, 1999), and this inhibition is reduced during the progression to AIDS (e.g., due to alterations in chemokine concentrations). Thus, the administration of human S100A8 and/or S100A9 as part of an HIV treatment regimen is contemplated to restore in part the integrity of the patient's immune system and/or to inhibit viral replication. Moreover, compounds such as wild type and mutant human S100A8 and S100A9 with chemokine inhibitory or fugetactic activity are contemplated to be suitable components of regimens designed to attenuate local immune responses (Sabroe et al., J Biol Chem, 275:25985-259921 2000). In fact, any immune response (e.g., asthma, glomerultonephritis, multiple sclerosis, etc.) mediated by RANTES and its receptors are contemplated to be susceptible to regulation by human S100A8 and/or S100A9.

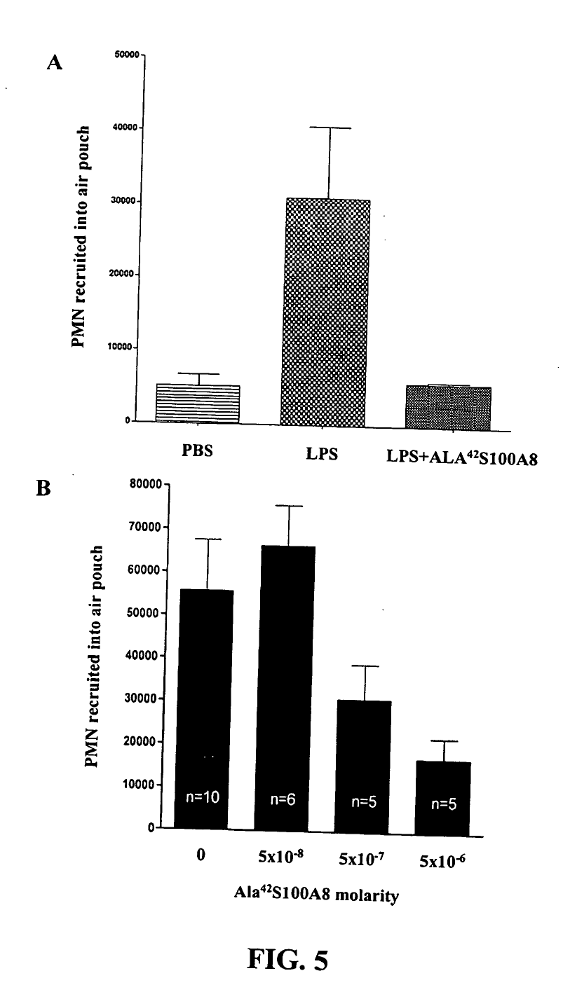

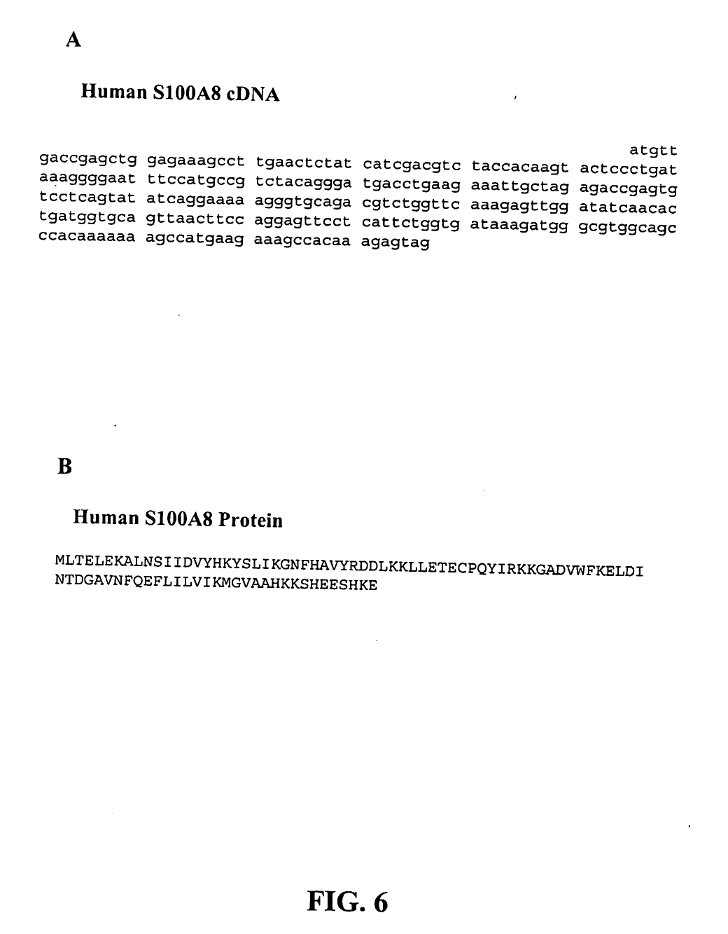

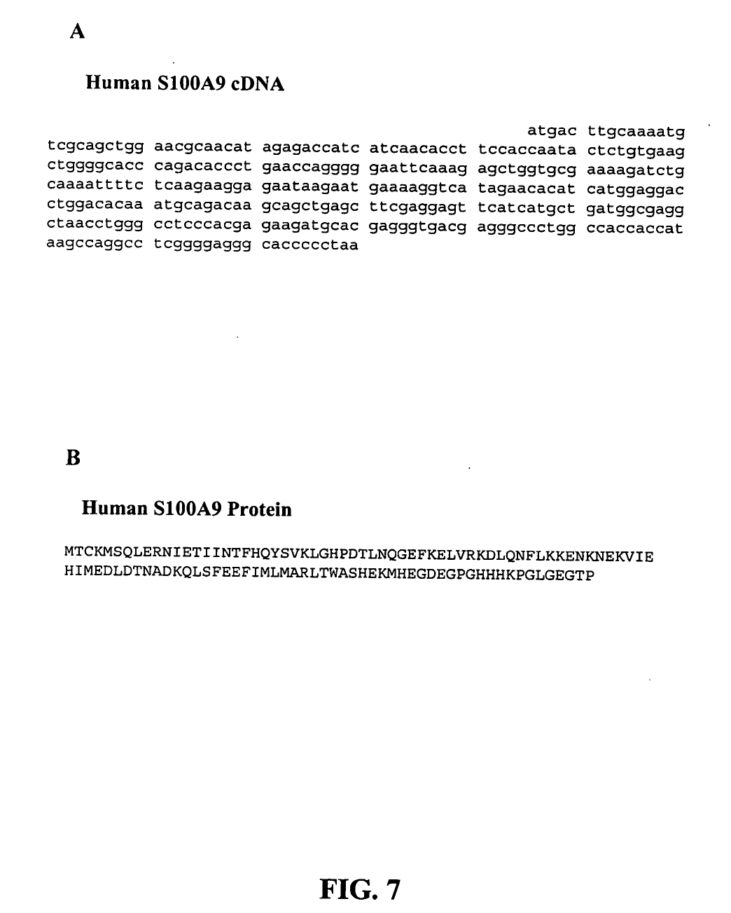

In a proof of concept study described herein in Example 7, and shown in FIG. 5, a mutant human S100A8 protein was shown to possess fugetactic activity in an in vivo rat air-pouch model. Specifically, Ala⁴²S100A8 abrogated the influx of neutrophils into air pouches in response to LPS. The inhibition of neutrophil influx by Ala⁴²S100A8 was also shown to be a dose-dependent process.

In addition, the present invention provides tools with which to isolate agonists and/or antagonists of human S100A8 and S100A9. In particular, the present invention provides methods comprising providing a chemokine receptor expressing cell, human S100A8 or A9, and a candidate compound, and contacting the cell with the human S100 protein in the presence and absence of the candidate compound. Candidate compounds which modulate (reduce or increase) S100 protein binding to the cells or which modulate S100-induced fugetaxis are contemplated to find use in various medicinal compositions

The inventors further contemplate many other uses of oxidation-resistant and oxidized human S100A8 and S100A9 in vitro and in vivo. The examples provided herein merely illustrate a range of methods and compositions for treatment of inflammatory disorders. One of ordinary skill in the art will appreciate that many other methods and compositions comprising human S100A8 and S100A9 can be used successfully.

### EXPERIMENTAL

The following examples are provided in order to demonstrate and further illustrate certain preferred embodiments and aspects of the present invention and are not to be construed as limiting the scope thereof.

In the experimental disclosure which follows, the following abbreviations apply: eq (equivalents); M (Molar); mM (millimolar); μM (micromolar); N (Normal); mol (moles); mmol (millimoles); μmol (micromoles); nmol (nanomoles); g (grams); mg (milligrams); μg (micrograms); ng (nanograms); l or L (liters); ml (milliliters); μl (microliters); cc (cubic centimeters); cm (centimeters); mm (millimeters); μm (micrometers); nm (nanometers); ° C. (degrees Centigrade); bp (base pair); kb (kilobase); WT (wild type); PM (peripheral monocytes); MCP1 (monocyte chemotactic protein); IL-8 (interleukin-8); PCR (polymerase chain reaction); Ab (antibody); mAb (monoclonal antibody); BME (2-mercaptoethanol) SDS (sodium dodecyl sulfate); MgCl₂ (magnesium chloride); NaCl (sodium chloride); EDTA (ethylene diamine tetraacetic acid); DEPC (diethyl pyrocarbonate); SSC (salt sodium citrate); BSA (bovine serum albumin); FCS (fetal calf serum); PBS (phosphate buffered saline); Tris (tris(hydroxymethyl) aminomethane); H₂O (water); IgG (immunoglobulin); Clonetics (Palo Alto Calif.); Invitrogen (Invitrogen Life Technologies, Carlsbad, Calif.); Sigma (Sigma Chemical Co., St. Louis, Mo.); Jackson (Jackson Laboratory, Bar Harbor, Me.); Boehringer Mannheim (Boehringer Mannheim, Indianapolis, Ind.); Fisher (Fisher Scientific, Pittsburgh, Pa.); Ambion (Ambion, Austin, Tex.); Vector (Vector Laboratories, Burlingame, Calif.); Zymed (Zymed Laboratories, South San Francisco, Calif.); Pharmingen (Pharmingen, San Diego, Calif.); Qiagen (Qiagen, Valencia, Calif.); Life Technologies (Life Technologies, Rockville, Md.); Alpha Innotec (Alpha Innotec, San Leandro, Calif.); and ATCC (American Type Culture Collection, Manassus, Va.).

### Example 1

### Recombinant Cytokines

Experiments were conducted with commercially available cytokines. S100A8 and S100A9 were initially purchased from BMA Biomedicals of Switzerland. The results from early experiments were corroborated with recombinant human S100 proteins produced in E. coli using a pGEX-2T GST vector according to standard protocols. The S100 proteins were kept at −20° C. in a solution consisting of 10 mM TRIS (pH 7.5), 0.1% cholate, 1 mM EDTA and 1 mM BME.

Mutant Human S100A8

The mutant human Ala⁴²S100A8 protein was produced as a GST fusion protein in a bacterial expression system after using standard site-directed mutagenesis methods to substitute the Cysteine at position 42 with an Alanine. Details of the techniques have been described previously (See, Harrison et al., J Biol Chem, 274:8561-8569, 1999). In addition, the production of additional mutant human S100A8 proteins is contemplated. In particular, mutant human S100A8 proteins comprising lysine substitutions at residues shown to undergo sulfinamide bond formation (See, Raftery et al., J Biol Chem, 276:33393-33401, 2001) are contemplated to find use in the compositions and methods of the present invention.

Mutant Human S100A9

The production of mutant human S100A9 proteins is accomplished using methods similar to those employed for the production of Ala⁴²S100A8. In particular, human S100A9 was shown to possess three functions regulated by oxidation: fugetaxis, anti-oxidant, and anti-bacterial. The oxidative modifications involve three methionine residues which are conserved in the mouse and rat proteins, located on the third and fourth alpha helices. The first Met residue is at position 61 on the third alpha helix, while the second and third are part of an “MLM” motif on the fourth helix. Multiple human S100A9 proteins have been produced as GST fusions, including: wild type (WT) S100A9, ALM, MLA, ALA, 61, 61ALM, 61MLA and 61ALA. The ALM mutant has a substitution of methionine 81 with alanine. The MLA mutant has a substitution of methionine 83 with alanine. The ALA mutant has substitutions of methionine 81 and 83 with alanine. The 61 mutant has a substitution of methionine 63 with alanine. The 61ALM mutant has substitutions of methionine 61 and 81 with alanine. The 61MLA mutant has substitutions of methionine 81 and 83 with alanine. The 61ALA mutant has substitutions of methionine 61, 81 and 83 with alanine. The A9z mutant has a truncation of the histidine-rich regions of the carboxyl tail.

Replacement of Met at position 61, with Ala yielded a protein which inhibits the oxidative burst function of neutrophils and monocytes, but does not display fugetactic functions. Oxidative modification of the Met residues of the MLM motif was found to control both the fugetactic and the anti-oxidant effect of S100A9. Specifically, ALM and ALA exhibit anti-oxidative function, whereas 61MLA exhibits fugetactic activity. Moreover, the different MLM mutants with a Met residue at position 61 (wild type) exhibit anti-bacterial activity.

### Example 2

### Effect of Human S100A8 and S100A9 on Leukocyte Movement in Under-Agarose Migration Assays

The effects of human S100A8 and S100A9 and of a control chemokine (CCL2/MCP1) on the movement of PM were first determined in an under-agarose migration system. The under-agarose migration assays were performed as previously described (Nelson et al., J Immunol, 115:1650-1656, 1975; and Foxman et al., J Cell Biol, 139:1349-1360, 1997). Peripheral monocytes (PM) were isolated on ficoll hypaque (Pharmacia) from blood drawn from healthy human volunteers and mixed with 10 to 20 units of sodium heparin. Cells were washed, re-suspended in PBS at a concentration of 200,000 cells/10 μl, and their viability (>95%) was assessed with tryptan blue in a hemocytometer. Using a plexiglass template and a sterilized blunted stainless steel biopsy punch, three wells were equidistantly bored in 1.2% agarose (GibcoBRL) diluted 1:1 with RPMI with 10% heat-inactivated fetal calf serum. Agarose plugs were removed with a pipette tip and vacuum. The central well was filled with 10 μl of PM (in PBS). Ten μl of a solution containing S100A proteins or control chemokines at various concentrations were added to a second well (experimental well). Control medium was added to the third well (negative control) on the opposite side from the experimental well. In several experiments, the PM were pre-incubated on ice for 30 min with a variety of antibodies or chemokines. Plates were incubated overnight at 37° C., 5% CO₂, then fixed with methanol for at least 30 min, followed by 47% buffered formaldehyde for at least 30 min. Plates were stained with 2% toluidine blue and left to air dry before being examined under a microscope. The distance of migration was measured and compared on both sides of the well under a microscope equipped with a gridded reticule. A migration ratio was calculated by dividing the distance traveled toward the experimental well by the distance traveled toward the negative well. Migration ratios greater than one indicate preferential migration toward a chemokine (chemotaxis), while migration ratios less than one indicate preferential migration away from a chemokine (fugetaxis).

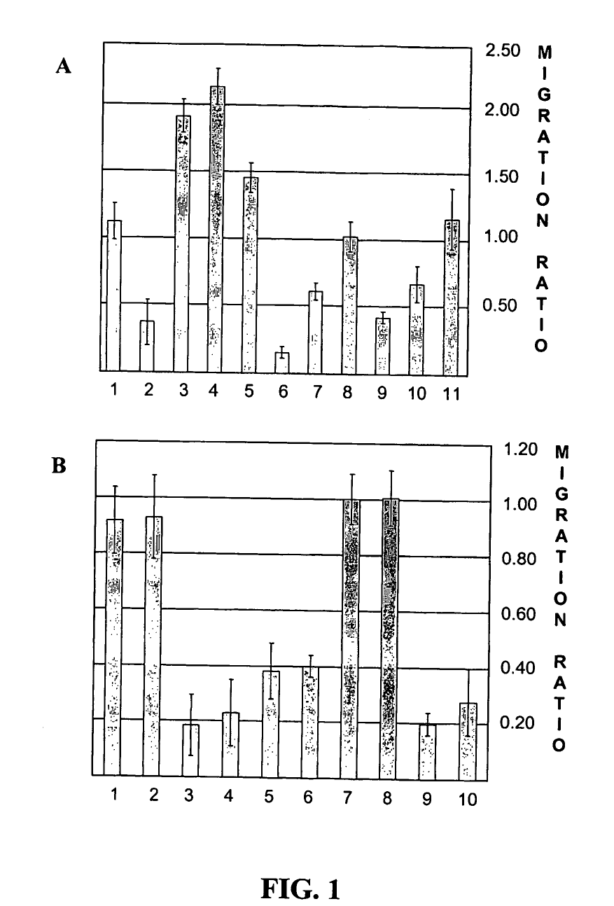

As shown in FIG. 1, panel A, the addition of S100A8 or S100A9 (0.01-1.0 picomol) elicited a consistent fugetaxis response that biased the migration of PM away from the S100 proteins. The S100 proteins did not exhibit a dual concentration-dependant chemotactic/fugetactic effect since at a lower concentration they elicited no chemotactic movement, even at concentrations at which murine S100A8 was shown to be chemotactic (Lackman et al., J Immunol, 150:2981-2991, 1993). In contrast, the control chemokine monocyte chemotactic protein (MCP1) exhibited a chemotactic response at 0.1, 1 and 10 picomol, and a fugetactic response at 1 nanomol. The fugetaxis of PM in under-agarose assays observed with MCP1 at a concentration greater than the chemotactic concentration is previously unreported.

The effect of the human S100A8 and S100A9 proteins on PM motility was shown to be pertussis toxin-sensitive indicating that it occurs at least in part through the activation of a G-protein coupled receptor. To confirm whether the S100 proteins signal through chemokine receptors, a receptor desensitization assay was employed in which chemokines with known receptor specificity were pre-incubated with PM in the central well of the under-agarose migration assay. In these assays, the fugetactic effect observed with 1 picomol of human S100A8 or S100A9 was tested in the presence and absence of control chemokines in the well containing the PM. As shown in FIG. 1, panel B, RANTES blocked the fugetactic activity of S100A8 and S100A9, whereas MCP1 and IL-8 had no effect. As heterologous receptor desensitization is thought to be restricted to N-formyl peptides and C5a (Foxman et al., J Cell Biol, 139:1349-1360, 1997; Foxman et al., J Cell Biol, 147:577-588, 1999; and Kitayama et al., J Immunol, 158:2340-2349, 1997), the inhibition of S100 by RANTES was considered not to be caused by RANTES cross-reactivity with an S100-specific receptor, but rather to be caused by the direct competition at a RANTES receptor. A role for RANTES receptors in S100-mediated fugetaxis was confirmed by the abrogation of fugetaxis by the addition of an anti-CCR3 mAb (7B11 described by Heath et al., J Clin Invest, 99:178-184, 1997), as depicted in FIG. 1, panel B, bars 9-12. However, later experiments revealed that 7B11 also interacted with the CCR1 receptor. Thus, during development of the present invention, the human S100A8 and S100A9 proteins were determined to act via CCR1, CCR3, or both. However, an understanding of the mechanism(s) is not necessary in order to make and use the present invention.

### Example 3

### Effect of Human S100A8 and S100A9 on Leukocyte Movement in Transwell Migration Assays and by Video Microscopy

A mouse lymphocyte cell line (4DE4) stably expressing human CCR1 or CCR3 was used to precisely define the interactions between the S100 proteins and the CC chemokine receptors. As lymphocytes are immobile in under-agarose assays (Nelson et al., J Immunol, 115:1650-1656, 1975), transwell migration studies were performed with S100A8, S100A9, the CCR3 ligand eotaxin, and the CCR1-specific ligand MIP1α, with IL8 and MCP1 (Baggiolini et al., Int J Immunopharmacol, 17:103-108, 1995) as negative controls. Stable CCR1- and CCR3-transfectants, were kindly provided by Dr. James Pease from the Leukocyte Biology Section, Imperial College School of Medicine in London, UK. The parental 4DE4 cell line was kindly provided by Dr. Philip Murphy from the Laboratory of Host Defenses, NIAID, National Institutes of Health in Bethesda, Md. The cell lines were maintained in RPMI 1640, 10% FCS, 1% non-essential amino acids (100×), 0.1% BME (1000×), 1% sodium pyruvate, and 1% glutamine. Approximately 0.1% G418 was added to the medium used to culture the two transfected cell lines.

The peripheral granulocytes used in these experiments were separated from EDTA-anticoagulated whole blood from healthy volunteers. Granulocytes were separated using Histopaque 119 and 1077, and red blood cells were lysed through hypotonic shock. Purified neutrophils were re-suspended in RPMI containing 0.5% BSA, at a concentration of 1 million cells/ml.

One hundred μl of RPMI containing one million 4DE4 cells or one hundred thousand leukocytes were placed in the upper chamber of a transwell apparatus (6.5 mm, 3 μm pore, polycarbonate membrane from Corning Costar Inc.), while various chemokines were placed in 600 μl RPMI in the lower well. The transwells were incubated at 37° C., 5% CO₂ for 3 and 5 hours for the leukocytes and for the 4DE4 cells, respectively. The cells which migrated through the upper chamber's filter to the lower chamber were collected and counted using flow cytometry and/or a hemocytometer. Migration ratios represent the ratio of the number of cells in the lower well to the number of cells in the lower well of the control (containing medium alone).

As shown in FIG. 2, panel A, S100A8 and S100A9 attracted CCR1-expressing cells to a greater extent than the control chemokine MIP1α in this system, whereas the S100 proteins induced a weaker chemotaxis in CCR3-expressing lymphocytes (which responded appropriately to eotaxin). Neither cell type reacted to IL-8 or MCP1. In a checkerboard experiment, S100A8 and A9 exhibited both a chemotactic and chemokinetic effect on cells expressing CCR1 or CCR3 as shown in FIG. 2, panel B. Thus, in contrast to the fugetactic effect of S100A8 and S100A9 on PM in under-agarose assays, these proteins attracted CCR1-transfected cells and CCR3-transfected lymphocytes to a lesser degree.

Chemotaxis of lymphocytes expressing CC receptors triggered by human S100A8 and S100A9 proteins are apparently inconsistent with the fugetactic effect of the same proteins observed in PM and neutrophils in under-agarose assays. These different cell responses in vitro are contemplated to be due to differences in assay conditions between transwell and under-agarose assays, and to inherent differences between PM, neutrophils and the CCR1/CCR3-transfected lymphocytes examined herein. To this end, transwell assays on PM and neutrophils were performed, which confirmed that S100A8 has a fugetactic effect on PM and neutrophils (See, FIG. 4, panel A). The fugetactic concentrations of the human S100A8 and S100A9 proteins of 10⁻⁹M in the transwell assays and the fugetactic concentration in the under-agarose assays (1 pmol) were similar to the levels observed in these two assays with classical chemokines such as IL-8 (Foxman et al., J Cell Biol, 139:1349-1360, 1997). Confirmation of the effect of S100 proteins on 4DE4 cells expressing CCR1 and CCR3 was also demonstrated by performing receptor internalization experiments (data not shown). However, an understanding of the mechanism(s) is not necessary in order to make and use the present invention.

The fugetactic response of peripheral neutrophils was also recorded by time-lapse digital video microscopy after neutrophils were plated in PBS containing various chemokines absorbed to a paper disk. Movement of the neutrophils away from the disk containing human S100A8 was clearly seen. As a negative control, a disk containing the PBS carrier solution displayed no effect, whereas the positive control fMLP (N-formyl-methionyl-leucyl-phenylalanine)-containing disk exhibited a chemoattractive effect on the neutrophils.

### Example 4

### Human S100A8 and S100A9 Induce Shape Changes in Granulocytes

Approximately 90 ul containing 90,000 peripheral granulocytes were added to 10 ul chemokines at a concentration of 10⁻⁸M. The samples were incubated for 4 min, transferred onto ice and fixed (Sabroe et al., J Biol Chem, 275:25985-25992, 2000). Flow cytometric analysis of the neutrophils showed (See, FIG. 4, panel C) that S100A8 and S100A9 induced increased forward scatter, consistent with a chemokine-induced shape change (Bryan et al., Am J Respir Crit Care Med, 165:1602-1609, 2002). The shape change and receptor internalization experiments confirmed that human S100A8 and S100A9 directly activate cells via their cell surface receptors. However, an understanding of the mechanism(s) is not necessary in order to make and use the present invention.

### Example 5

### Activity of Modified Human S100A8 and S100A9 In Vitro

As described above, PM and neutrophils exclusively respond with fugetaxis to as little as nanomolar ranges of the human S100A8 and S100A9 proteins, a concentration that is substantially lower than the nanomolar-range concentration in normal human serum (Lugering et al., Digestion, 56:406-414, 1995). Given that epithelium expresses S100A8 and S100A9 in a variety of inflammatory conditions and that these proteins are fugetactic to PM and neutrophils, it is contemplated that this would create a barrier to the inflammatory response. Interestingly, S100A8 and S100A9 have also been reported to constitute upward of 40% of the total neutrophil cytoplasmic protein content (Edgeworth et al., J Biol Chem, 266:7706-7713, 1991). Thus, because of the very low concentrations at which S100A8 and A9 exert a fugetactic effect, their functions are contemplated to be tightly regulated and at least in part controlled by post-translational modifications of the proteins.

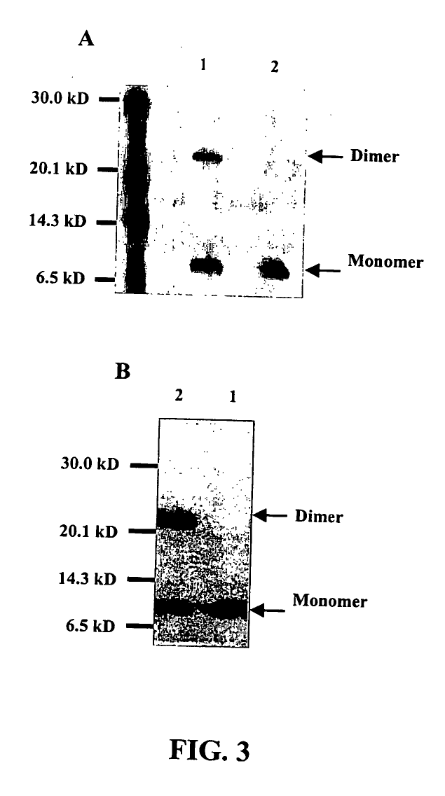

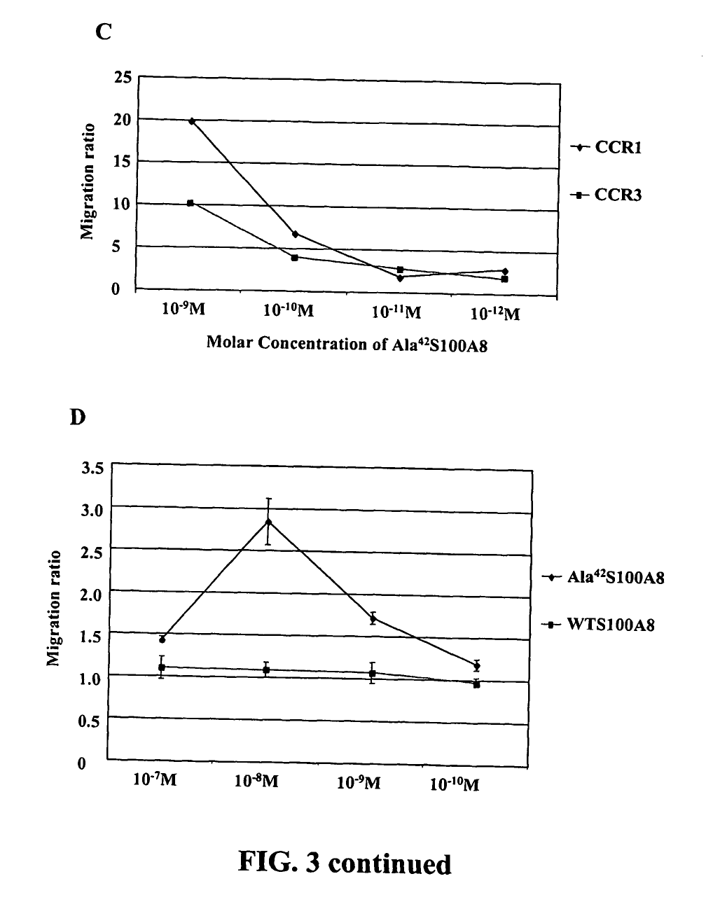

Formation of disulfide bridges between conserved cysteine residues in murine S100A8 has been shown to regulate its function (Harrison et al., J Biol Chem, 274:8561-8569, 1999). Oxidative modification of human S100 proteins by neutrophils through the action of myeloperoxidase is contemplated to serve as a counter-fugetaxis mechanism. Thus, in order to confirm the effect of oxidation on the functions of human S100A8, a mutant protein was prepared. Specifically, the cysteine residue at position 42 was mutated to an alanine (Ala⁴²S100A8). The overall structure of human S100A8 was not destroyed by the mutation, as a mAb directed against the wild type S100A8 protein also detected Ala⁴²S100A8 in a western blot (See, FIG. 3, panel B). Importantly, the Ala⁴²S100A8 mutant also retained fugetactic activity for peripheral neutrophils in transwell migration assays (See, FIG. 4, panel B). However, the mutant protein was unable to form a covalently-bound homodimer of S100A8, even after treatment with 100 uM sodium hypochlorite for 15 min (See, FIG. 3, panel A). In contrast to wild type S100A8, the Ala⁴²S100A8 mutant retained its ability to chemo-attract CCR1- and CCR3-expressing cell lines, independent of its oxidative state, and at concentrations comparable to that observed with wild type S100A8. Moreover, although the oxidized wild type S100A8 protein was ineffective at promoting chemotaxis, it retained the ability to block attraction of CCR3-transfected cells by eotaxin (See, FIG. 2, panel C). In addition, the Ala⁴²S100A8 mutant remained fugetactic for neutrophils even while oxidized, whereas the fugetactic activity of the wild type S100A8 protein for neutrophils was destroyed by oxidation (See, FIG. 3, panel D). However, an understanding of the mechanism(s) is not necessary in order to make and use the present invention.

### Example 6

### Effects of Human S100A8 and S100A9 on Leukocyte Migration In Vivo in a Mouse Peritoneal Model

The fugetactic activity of human S100A8 and S100A9 is assessed in vivo in C57BL/6J mice (Jackson Laboratories, Bar Harbor, Me.) as described in U.S. Pat. No. 6,488,054, herein incorporated by reference. Briefly, the mice are primed subcutaneously with 100 kg chicken ovalbumin (OVA) in 100 μl of Complete Freund's Adjuvant (CFA). The OVA used herein contains an insignificant amount of lipopolysaccharide (less than 0.01%) LPS. Three days later OVA-primed mice are challenged by intraperitoneal (IP) injection with 100 μg of OVA dissolved in 250 μl of double distilled water. A second 250 μl IP injection is given to these mice the following day, which contains human S100A8, human S100A9 or a control recombinant protein, at a concentration at which the human proteins are shown to have fugetactic activity in vitro. The mice are euthanized 3 and 24 hours following the second IP injection and a peritoneal lavage with 5 mls of PBS is performed with direct visualization of the peritoneal sac and its contents in order to avoid peripheral blood contamination. Peritoneal fluid cells harvested in this way should contain less than 0.1% red blood cells.

Three groups of control mice are used. In the first, C57BL/6J mice are euthanized prior to OVA priming, and a peritoneal lavage is performed. The second group of control mice are primed subcutaneously and challenged with OVA intraperitoneally as above and then euthanized 3 and 24 hours later. The third group are primed and challenged with OVA as above and injected intraperitoneally with 250 μl of water alone 24 hours later. These mice are euthanized and a peritoneal lavage is performed after a further 3 and 24 hours.

Cells harvested from the peritoneal fluid are counted with a hemocytometer and their viability determined by trypan blue exclusion and the total viable nucleated cell count/ml of peritoneal fluid is determined. Hematological smears of peritoneal fluid are also prepared and stained with Giemsa. Flow cytometry is then done on the peritoneal fluid cells using mAbs-reactive with leukocyte lineage-specific antigens (e.g., CD3, CD4, CD8, etc). It is contemplated that the human S100A8 and S100A9 protein treatments are suitable for abrogation of a leukocyte infiltrate induced by the IP challenge with OVA, whereas the control protein is not expected to significantly effect leukocyte emigration.

As an alternative to induction of a leukocyte infiltrate with OVA in adjuvant, the administration of other irritants or inflammatory compositions is contemplated either coincidentally or prior to S100A8 and S100A9 administration.

### Example 7

### Effects of Modified Human S100A8 on Leukocyte Migration In Vivo in a Rat Air-Pouch Model

The fugetactic activity of mutant human S100A8 was assessed in vivo in rats as described (Cartmell et al., J Physiol, 531:171-180, 2001; and Matsukura et al., J. Rheumatol, 25:539-545, 1998; herein incorporated by reference in their entirety). Briefly, rats were anesthetized with 2-3% isoflurane in oxygen. Their backs were shaved and swabbed with 70% ethanol. Then 20 ml of sterile air (passed through a 0.2 μm filter) was injected subcutaneously to form an air pouch. Three days later, this procedure was repeated, except that only 10 ml of sterile air was injected. Rats were used for assessment of leukocyte recruitment 3 days following the second air injection.

In order to harvest leukocytes, the rat 7-day old air pouches were injected with 300 μl LPS (30 ng) or sterile PBS in the presence or absence of Ala⁴²S100A8. Three hours later, the rats were anesthetized with pentobarbital (65 mg/kg) and cells were collected by injecting 5 ml of sterile PBS into the pouch. After gentle massaging of the pouch, the cell-containing fluid was aspirated, placed into sterile culture tubes, and centrifuged at 1500 rpm for 10 min (25° C.). Supernatant was aspirated and the cells were resuspended in 1 ml room temperature PBS containing 1% bovine serum albumin. Ten μl of 1 mg/ml Hoechst 33342 (bisbenzimide) solution, was added to flow cytometry tubes in the dark, to aid in separating nucleated cells from red blood cells. Bisbenzimide was incubated with the sample for at least 40 min in the dark. One μl of 1 mg/ml propidium iodide solution was added immediately to assess nucleated cell viability before flow cytometry was performed.

Leukocytes in the air pouch samples were quantified by flow cytometry using nuclear content, forward scatter and side scatter patterns obtained from excitation at 488 nM and 354/63 nM wavelengths. A FITC labeled rabbit anti-rat PMN antibody (catalog # AIFAD51140 from Accurate Chemical and Scientific Corporation, Westbury, N.Y.) was used to quantify the number of PMN recruited to the air-pouch. Log fluorescence was measured for 30 sec at constant pressure for each sample, using a triple laser Vantage SE cell sorter (Becton Dickinson, San Jose, Calif.). Data acquisition was done using CellQuest Pro software, version 4.01 (Becton Dickinson), and off-line analysis was performed using FloJo, version 4.5 (Tree Star, Inc.).

### Example 8

### Human S100A8 and S100A9 Inhibit Eotaxin-Induced Chemotaxis of CCR3-Transfected Cells

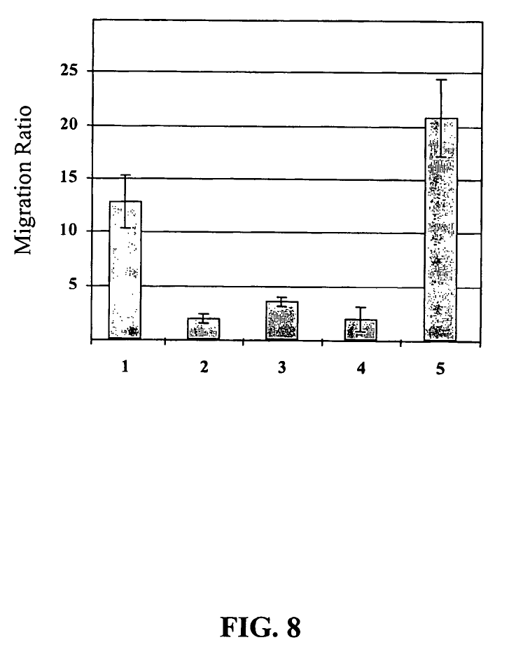

Eotaxin is a CC chemokine specific for the chemokine receptor CCR3. This receptor is expressed by a number of leukocytes including eosinophils, basophils, mast cells and a subset of lymphocytes having a Th2 cytokine expression pattern. As shown herein in FIG. 8, wild type human S100A8 and S100A9 inhibit the CCR3 mediated chemotactic effect of eotaxin. In particular, S100A8 and S100A9 inhibited the in vitro migration of a murine lymphoma cell line (4DE4) stably transfected with the CCR3. Thus, S100A8 and S100A9 are contemplated be suitable therapeutic proteins for the treatment of conditions associated with tissue eosinophilia (e.g., asthma, atopic dermatitis etc.).

### Example 9

### Wild Type and Modified Human S100A8/A9 Complexes Inhibit Growth of Candida albicans

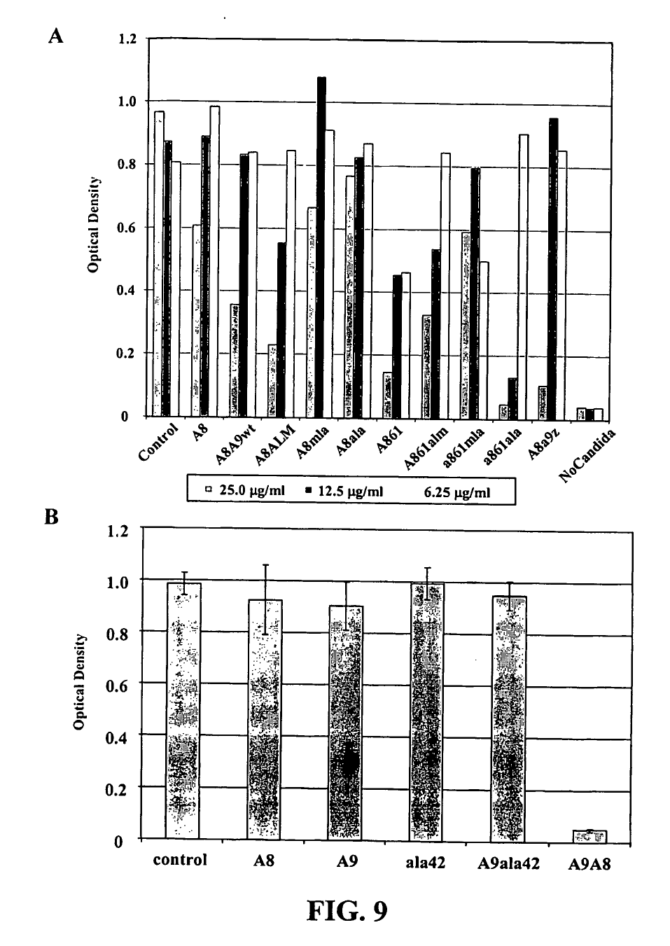

This example describes the use of S100A8/A9 complexes to inhibit growth of the model fungal organism, Candida albicans. Briefly, about 1000 yeast were incubated overnight at 30° C. in the presence of various concentrations of wild type and mutant complexes. Growth of yeast was quantified spectrophotometrically by measuring the optical density at a wavelength of 600 nm. As shown herein, the post-translational modification(s) of the proteins was shown to have an important effect on the anti-fungal effect observed in vitro. Cysteine at residue 42 of S100A8, and methionines at residues 63, 81 and 83 of S100A9 were found to display a regulatory function over the observed anti-fungal effect (See, FIG. 9). In addition, the polyhistidine domains (e.g., divalent cation binding sites) in the C-terminus of both proteins are contemplated to contribute to the antifungal activity.

As described in more detail in Example 1 above, several mutants have been generated including S100A8 with a mutation of cysteine at residue 42, combinatorial mutations of each of the three methionine residues in S100A9, as well as truncation of the divalent cation binding domains in both proteins. Some of the mutations described herein, are contemplated to possess therapeutic value in vivo. In addition, the methods described herein are contemplated to be suitable for generation of additional S100A9 mutants with even more potent anti-fungal activities.

### Example 10

### Mutant Human S100A9 Inhibits Pseudomonas aeruginosa Growth Induced by Wild Type S100A9 or a S100A8/A9 Complex

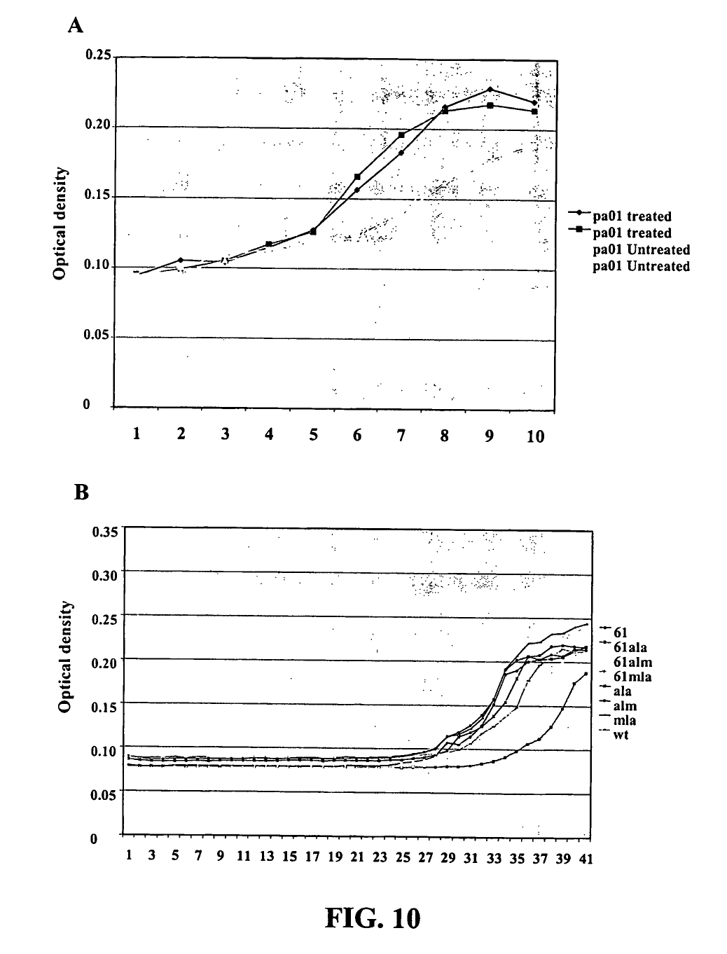

This example describes the use of S100A9 and S100A8/A9 complexes to modulate growth of Pseudomonas aeruginosa. In particular, S100A9 by itself or complexed to S100A8 was found to enhance the growth of Pseudomonas aeruginosa in vitro, as shown in FIG. 10. The 61ALA mutant of S100A9 does not induce this enhanced growth, and even competes with the wild type protein. Thus, modified S100A9 proteins are contemplated to be suitable therapeutics for Pseudomonas-associated pneumonia. Treatment with mutant S100A9 proteins is contemplated to be especially beneficial for treatment of Pseudomonas-induced pneumonia in patients with cystic fibrosis, as these patients have high levels of calprotectin (wild type S100A8/A9 complexes) in their pulmonary secretions. The mutants could be used alone or in conjuction with standard anti-pseudomonal antibiotics. In addition, the ALA⁴²S100A8 mutant is also contemplated to act as an inhibitor of the wildtype S100A9 and S100A8/A9-induced growth of Pseudomonas.

In short, S100A8/A9 enhancement of Pseudomonas growth is contemplated to be partly responsible for the recurrent Pseudomonas infections observed in cystic fibrosis patients. The ALA⁴²S100A8 and/or the 61ALAS100A9 proteins are contemplated to be suitable therapeutic proteins against Pseudomonas lung infection on their own, or in-conjunction with other available therapeutics.

### Example 11

### S100A8 and S100A9 Inhibition of Casein Kinase II and HPV-Transformed Cell Growth

The human papillomavirus (HPV) type 16 E7 protein is a major viral oncoprotein that interacts with a number of host intracellular proteins of growth-regulatory pathways. HPV16 E7 is phosphorylated by casein kinase II (CKII) and E7 phosphorylation is contemplated to regulate its interaction with intracellular ligands. Two S100 family calcium-binding proteins, S100A8 and S100A9 form a protein complex (S100A8/A9, also known as MRP-8/14), that inactivates CKII. The inhibitory effect of the MRP-8/14 complex on CKII activity and HPV16 E7 phosphorylation was examined. Both CKII activity and HPV16 E7 phosphorylation was inhibited by uptake of exogenous MRP-8/14 and activation of endogenous MRP-8/14. Analysis of MRP protein expression in primary keratinocytes and in HPV16 and HPV18-transformed cervical and foreskin epithelial cell lines, showed that expression of MRP-8, MRP-14 and the MRP-8/14 complex was detected only in primary untransformed keratinocytes, but not in the HPV-infected immortalized epithelial cells. CKII activity in HPV-immortalized keratinocytes was approximately 4-fold higher than in HPV-negative primary keratinocytes. Treatment of HPV-positive immortalized epithelial cells with exogenous MRP-8/14 showed complete inhibition of cell growth within two weeks, compared with HPV-negative primary and immortalized cervical epithelial cells which showed 25% and 40% growth inhibition, respectively. MRP-8/14 mediated growth inhibition in HPV-infected cervical epithelial cells is contemplated to be due at least in part to E7 hypophosphorylation, leading to loss of E7-mediated growth stimulation. Together these results indicate that the MRP-8/14 protein complex in HPV-infected epithelial cells plays an important role in regulation of CKII-mediated E7 phosphorylation, and in inhibition of HPV's oncogenic activity. In addition, the restoration of sufficient MRP-8/14 levels in HPV-infected cells is contemplated to induce lesion regression, and the progression to cancer.

In addition, MRP-8/14 through inhibition of casein kinase II, is contemplated to inhibit the release of IκB from NF-κB, thereby inhibiting the life cycle of HIV. MRP-8 and/or MRP14 are also contemplated to inhibit HIV replication as competitive inhibitors of HIV co-receptors such as CCR3. Finally MRP-8 and/or MRP-14 binding to CCR5 and CCR3 on host lymphocytes is contemplated to stimulate cell-mediated immune responses to HIV.

In summary, the present invention provides numerous advances and advantages over the prior art, including methods and compositions for the alleviating inflammation. All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention, which are obvious to those skilled in diagnostics, cell culture, and/or related fields, are intended to be within the scope of the present invention.

We claim:

1. A method comprising:
a) providing;
i) at least one leukocyte, and
ii) a composition comprising a human S100A8 or S100A9 protein; and
b) contacting said leukocyte with said composition under conditions suitable for repelling said leukocyte.

2. The method of claim 1, wherein said leukocyte is selected from the group consisting of a monocyte, a neutrophil and an eosinophil.

3. The method of claim 1, wherein said leukocyte expresses at least one chemokine receptor selected from the group consisting of CCR1, CCR3 and CCR5.

4. The method of claim 1, wherein said protein comprises at least one mutation inhibiting post-translational modification of said protein.

5. The method of claim 4, wherein said inhibiting post-translational modification comprises conferring oxidation resistance to said protein.

6. The method of claim 4, wherein said mutation further prevents dimerization of said protein.

7. A method comprising:
a) providing;
i) a subject with one or more symptoms of inflammation; and
ii) a composition comprising a human S100A8 or S100A9 protein; and
b) administering said composition to said subject under conditions such that at least one of said symptoms is reduced or eliminated.

8. The method of claim 7, wherein said subject has an inflammatory disorder selected from the group consisting of allergy, asthma, artherosclerosis, atopic dermatitis, autoimmune disease, cystic fibrosis, infection, injury, meningitis, psoriasis, and transplant rejection.

9. The method of claim 8, wherein said infection is with a microorganism selected from the group consisting of Candida albicans, Pseudomonas aeriginosa, human papillomavirus-16, and human immunodeficiency virus type 1.

10. The method of claim 7, wherein said one or more symptoms is selected from the group consisting of pain, heat, redness and swelling.

11. The method of claim 10, wherein said swelling comprises a leukocyte infiltrate.

12. The method of claim 11, wherein said leukocyte infiltrate comprises a cell selected from the group consisting of a monocyte, a neutrophil and an eosinophil.

13. The method of claim 7, wherein said protein comprises at least one mutation inhibiting post-translational modification of said protein.

14. The method of claim 13, wherein said inhibiting post-translational modification comprises conferring oxidation resistance to said protein.

15. The method of claim 13, wherein said mutation further prevents dimerization of said protein.

16. A composition comprising a nucleic acid sequence encoding a mutant human S100A8 protein that is at least 70% identical to SEQ ID NO:2 or a mutant human S100A9 protein that is at least 70% identical to SEQ ID NO:4, wherein said nucleic acid sequence comprises at least one mutation inhibiting post-translational modification of said protein.

17. The composition of claim 16, wherein said inhibiting post-translational modification comprises conferring oxidation resistance to said protein.

18. The composition of claim 16, wherein said mutation further prevents dimerization of said protein.

19. The composition of claim 16, wherein said mutation results in an amino acid substitution of a cysteine, a lysine or a methionine residue, and wherein said mutation does not destroy leukocyte-repellent activity of said protein.

20. The composition of claim 19, wherein said amino acid substitution comprises a replacement of Cysteine at residue 42 with an Alanine in said human S100A8 protein.

21. The composition of claim 19, wherein said amino acid substitution comprises a replacement of Methionine at one or more of residue 61, residue 81, and residue 83, with an Alanine in said human S100A9 protein.

22. A composition comprising a mutant human S100A8 protein that is at least 70% identical to SEQ ID NO:2 or a mutant human S100A9 protein that is at least 70% identical to SEQ ID NO:4, wherein said protein comprises at least one mutation inhibiting post-translational modification of said protein.

23. The composition of claim 22, wherein said inhibiting post-translational modification comprises conferring oxidation resistance to said protein.

24. The composition of claim 22, wherein said mutation further prevents dimerization of said protein.

25. The composition of claim 22, wherein said mutation results in an amino acid substitution of a cysteine, a lysine or a methionine residue, and wherein said mutation does not destroy leukocyte-repellent activity of said protein.

26. The composition of claim 25, wherein said amino acid substitution comprises a replacement of Cysteine at residue 42 with an Alanine in said human S100A8 protein.

27. The composition of claim 25, wherein said amino acid substitution comprises a replacement of Methionine at one or more of residue 61, residue 81, and residue 83, with an Alanine in said human S100A9 protein.


SEQUENCE LISTING (4 sequences)

SEQ ID NO: 1 - DNA, 282 bp, Homo sapiens
atgttgaccgagctggagaaagccttgaactctatcatcgacgtctaccacaagtactccctgataaaggggaatttccatgccgtctacagggatgacctgaagaaattgctagagaccgagtgtcctcagtatatcaggaaaaagggtgcagacgtctggttcaaagagttggatatcaacactgatggtgcagttaacttccaggagttcctcattctggtgataaagatgggcgtggcagcccacaaaaaaagccatgaagaaagccacaaagagtag

SEQ ID NO: 2 - protein, 93 aa, Homo sapiens
MLTELEKALNSIIDVYHKYSLIKGNFHAVYRDDLKKLLETECPQYIRKKGADVWFKELDINTDGAVNFQEFLILVIKMGVAAHKKSHEESHKE

SEQ ID NO: 3 - DNA, 345 bp, Homo sapiens
atgacttgcaaaatgtcgcagctggaacgcaacatagagaccatcatcaacaccttccaccaatactctgtgaagctggggcacccagacaccctgaaccagggggaattcaaagagctggtgcgaaaagatctgcaaaattttctcaagaaggagaataagaatgaaaaggtcatagaacacatcatggaggacctggacacaaatgcagacaagcagctgagcttcgaggagttcatcatgctgatggcgaggctaacctgggcctcccacgagaagatgcacgagggtgacgagggccctggccaccaccataagccaggcctcggggagggcaccccctaa

SEQ ID NO: 4 - protein, 114 aa, Homo sapiens
MTCKMSQLERNIETIINTFHQYSVKLGHPDTLNQGEFKELVRKDLQNFLKKENKNEKVIEHIMEDLDTNADKQLSFEEFIMLMARLTWASHEKMHEGDEGPGHHHKPGLGEGTP